(19) United States
(12) Patent Application Publication
(10) Pub. No.: US 2020/0061018 A1
(43) Pub. Date: Feb. 27, 2020

(54) METHODS AND COMPOSITIONS FOR TREATING PARKINSON'S DISEASE

(71) Applicant: Cove Bio LLC, Park City, UT (US)

(72) Inventors: Matthew Carl Lawyer, Park City, UT (US); Carl Henry Lawyer, Park City, UT (US)

(21) Appl. No.: 16/439,164

(22) Filed: Jun. 12, 2019

Related U.S. Application Data
(63) Continuation-in-part of application No. PCT/US17/66218, filed on Dec. 13, 2017.
Application No. PCT/US17/66218 is a continuation-in-part of application No. PCT/US18/24892, filed on Mar. 28, 2018.
(60) Provisional application No. 62/433,324, filed on Dec. 13, 2016.
(60) Provisional application No. 62/504,898, filed on May 11, 2017.

(51) Int. Cl. A61K 31/337; A61K 31/536; A61P 25/16; A61P 29/00; A61K 9/00; A61N 5/06

(52) U.S. Cl. CPC A61K 31/337; A61K 31/536; A61N 5/062; A61P 29/00; A61K 9/0014; A61P 25/16

(57) ABSTRACT

Provided herein are methods and compositions for treating, mitigating, or preventing Parkinson's disease by administration of an anti-malassezial agent wherein the anti-malassezial agent is oteseconazole, tavaborole or 2-(3,5-dimethyl-1H-pyrazol-1-yl)-5-methylphenol.

### RELATED APPLICATIONS

This application is a continuation-in-part of International Application No. PCT/US17/66218, which designated the United States and was filed on Dec. 13, 2017, published in English, which claims the benefit of U.S. Provisional Application No. 62/433,324, filed on Dec. 13, 2016, and U.S. Provisional Application No. 62/504,898, filed on May 11, 2017. This application is a continuation-in-part of International Application No. PCT/US18/24892, which designated the United States and was filed on Mar. 28, 2018, published in English. The entire teachings of the above applications are incorporated herein by reference.

### TECHNICAL FIELD

The present invention is directed to the fields of pharmaceutical science and clinical medicine. Various embodiments relate generally to methods and compositions for the prevention, mitigation, or treatment of Parkinson's disease.

### SUMMARY OF THE INVENTION

Disclosed herein are methods and compositions for diagnosing, treating, mitigating, or preventing Parkinson's disease based on the unexpected discovery that Malassezia infection is a cause of Parkinson's disease. In particular, provided herein are methods and compositions for preventing, mitigating, or treating Parkinson's disease by administering to a subject in need thereof an effective amount of an anti-malassezial agent.

Also provided are methods and compositions for killing or inhibiting the growth of Malassezia fungi to treat, mitigate, or prevent Parkinson's disease.

In some embodiments, the anti-malassezial agent may be any substance that has a biocidal and/or biostatic activity for Malassezia fungi.

In some embodiments, the anti-malassezial agent is an antifungal compound selected from the group consisting of zinc pyrithione, ciclopirox olamine, a polyene (e.g., amphotericin B), an azole (e.g., ketoconazole, itraconazole, fluconazole, and voriconazole), an allylamine (e.g., terbinafine), a morpholine (e.g., amorolfine), a sulfonamide (e.g., sulfacetamide), a glucan synthesis inhibitor or any combination thereof.

The antifungal agent is preferably a lipase inhibitor. Preferably the lipase inhibitor is orlistat or cetilistat, or any combination thereof.

The antifungal compound may be in the form of a topical agent, an intranasal agent, an oral agent, a systemic agent, an antimetabolite and mixtures thereof.

In some embodiments, the anti-malassezial agent is a debridement compound, such as a wash solution. Preferably the debridement compound is a hypertonic saline solution.

In some embodiments, the anti-malassezial agent is an anti-inflammatory compound. Preferably the anti-inflammatory agent is ibuprofen or betamethasone.

In some embodiments, the anti-malassezial agent is an antiseptic compound. In further embodiments, the antiseptic compound is a laundry composition.

In some embodiments, the anti-malassezial agent is a UV light therapy.

In one aspect, the present invention relates to pharmaceutical compositions for the treatment, mitigation, or prevention of Parkinson's disease comprising an anti-malassezial agent.

In another aspect, the present invention relates to methods for treatment, mitigation, or prevention of Parkinson's disease, comprising administering to a patient in need thereof a pharmaceutical composition of the invention. Some preferred embodiments comprise pharmaceutical compositions for treating Parkinson's disease, comprising a therapeutically effective amount of an anti-malassezial agent and a pharmaceutically acceptable carrier.

It is understood that aspects and embodiments of the invention described herein include “consisting” and/or “consisting essentially of” aspects and embodiments. Further aspects and embodiments of the invention are set forth below. These and other aspects will be readily apparent to the skilled artisan in light of disclosure as a whole.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 shows a graphical depiction of homology of leucine-rich repeats between Malassezia and the human LRRK2.

FIG. 2 shows a graphical depiction of the Malassezial enzyme gene sequences for myxalamid polyketide synthase MxaB and epothilone polyketide synthase D.

FIG. 3 shows a graphical depiction of the homology of polyketide synthetase genes between Malassezia and Beauveria.

FIG. 4 Shows a graphical depiction of the phylogenetic tree of relative evolutionary branching among various fungi discussed in the present disclosure.

### DETAILED DESCRIPTION OF THE INVENTION

Unless defined otherwise, all technical and scientific terms used herein have the same meaning as commonly understood by one of ordinary skill in the art to which the present disclosure belongs.

It is understood that the present disclosure is not limited by the particular embodiments disclosed herein, and any methods, components, materials, etc., similar or equivalent to those described herein can be used in the testing or practice of embodiments of the present disclosure.

Where a range of values is disclosed herein, it is understood that numeric ranges are inclusive of the numbers defining the range and that each intervening value, to the tenth of the unit of the lower limit unless the context clearly dictates otherwise, between the upper and lower limits of that range is also specifically disclosed. It is also understood that each smaller range between any stated value or intervening value in a stated range and any other stated or intervening value in the stated range is encompassed within the present disclosure.

As used herein, the singular forms “a”, “an”, and “the” include plural referents unless the context clearly dictates otherwise. The term “and/or” includes any and all combinations of one or more of the associated listed items.

### I. Scientific Discovery

The present invention is based on the inventors surprising and unexpected discovery that Malassezia is a cause of Parkinson's disease.

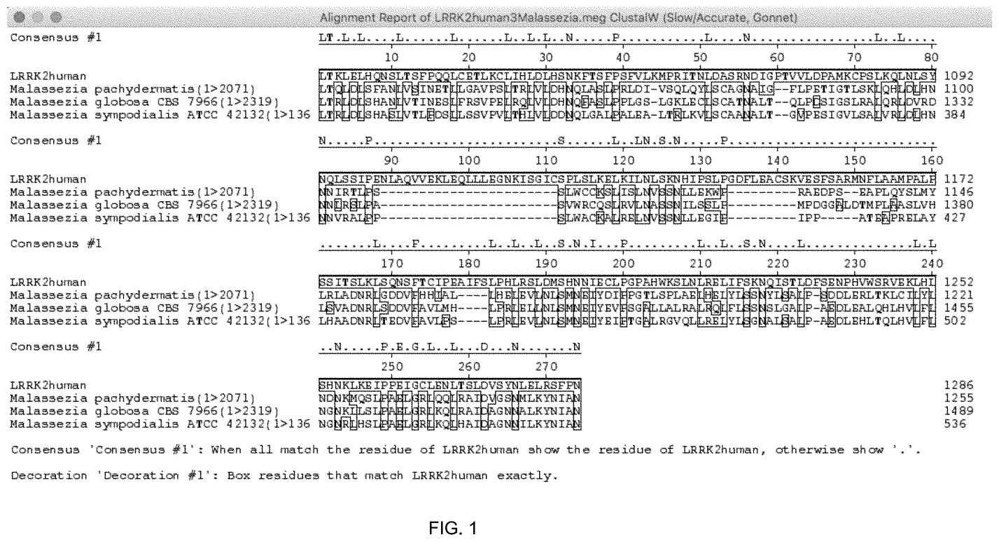

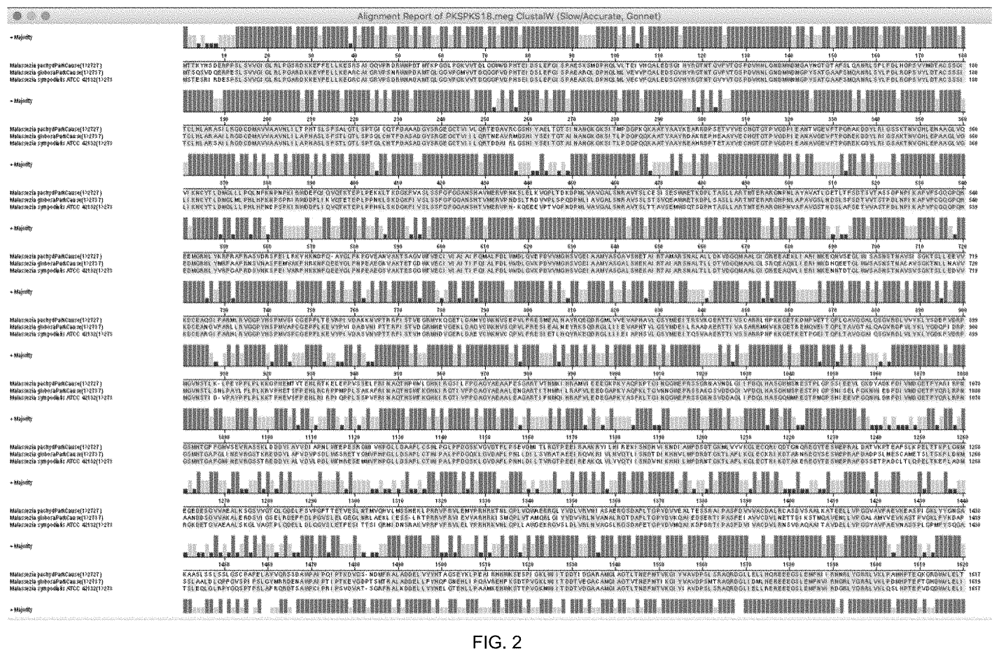

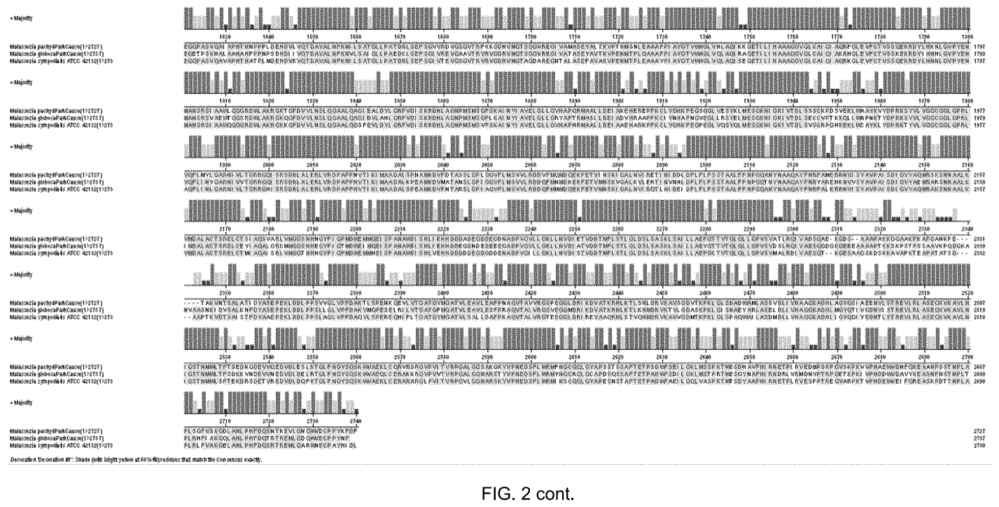

While not wishing to be bound by theory it is believed that Malassezia produce brain penetrant mycotoxins that cause apoptosis and cell death of the nigral dopaminergic neurons (nigral melanocytes) located in the substantia nigra of the midbrain. Reduction of these neurons is a known cause of attenuation in striatal dopamine efflux correlating positively with increased severity of Parkinson's disease. The inventors performed comparative genomic analyses of Malassezia species and other sources of human fungal infection. Careful examination of the results of their analyses led to their astonishing discovery that Malassezial enzymes that produce Malassezial brain-penetrant mycotoxins are encoded and expressed in the genomes of Malassezia known to infect the human skin (epidermis) as well as the human gastrointestinal tract (endodermis). For example, the genomes of Malassezia globosa, Malassezia pachyderma, and Malassezia sympodialis have been discovered by the inventors to each contain the genes for myxalamid polyketide synthase MxaB and epothilone polyketide synthase D, enzymes that produce brain-penetrant mycotoxins epothilone D and myxalamid. The brain-penetrant mycotoxin epothilone D destabilizes cellular microtubule function of neurons. Reduced microtubule stability in nigral neurons is known to be associated with Parkinson's disease. The brain-penetrant mycotoxin myxalamid inhibits respiratory electron transport chain function at mitochondrial complex 1. Inhibition of respiratory electron transport chain function at mitochondria complex 1 in nigral neurons is a known to be associated with Parkinson's disease. Malassezia produces and secretes malassezin, which is a brain-penetrant mycotoxin known to cause apoptosis and cell death in human primary epidermal melanocytes. Nigral melanocytes share the same embryological precursor origin (i.e., neural crest cells) as these epidermal melanocytes. Parkinson's disease has a known positive correlation between severity of disease and cell death of the pigmented nigral neurons in the midbrain. Myxobacteria are known to produce and secrete mycotoxins that have human cell toxicity. For example, Sorangium cellulosum produces and secretes Ixabepilone, an epothilone analog that is commercially manufactured and sold under the brand name IXEMPRA by Bristol-Myers Squibb (New York, N.Y.) for use as an anti-neoplastic drug (chemotherapy) for breast cancer cells Leucine-rich repeat kinase 2 (LRRK2) is an enzyme that in humans is encoded by the PARK8 gene and is a member of the leucine-rich-repeat kinase family of genes. Variants of the human LRRK2 gene are associated with an increased risk of acquiring Parkinson's disease. People with pathogenic LRRK2 mutations (e.g., G2019S, R1441G, Y1699C, R114C, R1441H, and I2020T) are at increased risk of acquiring Parkinson's disease as compared to the general population. The inventors have discovered that Malassezia genes encode homologous leucine-rich repeats in malassezial proteins as found in the human LRRK2 gene (FIG. 1).

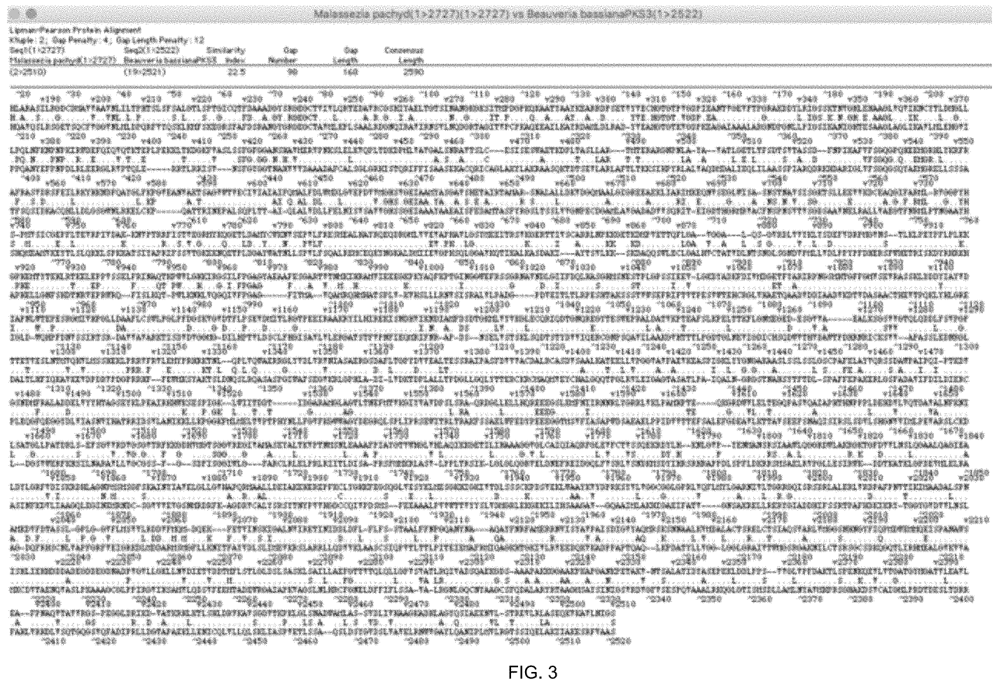

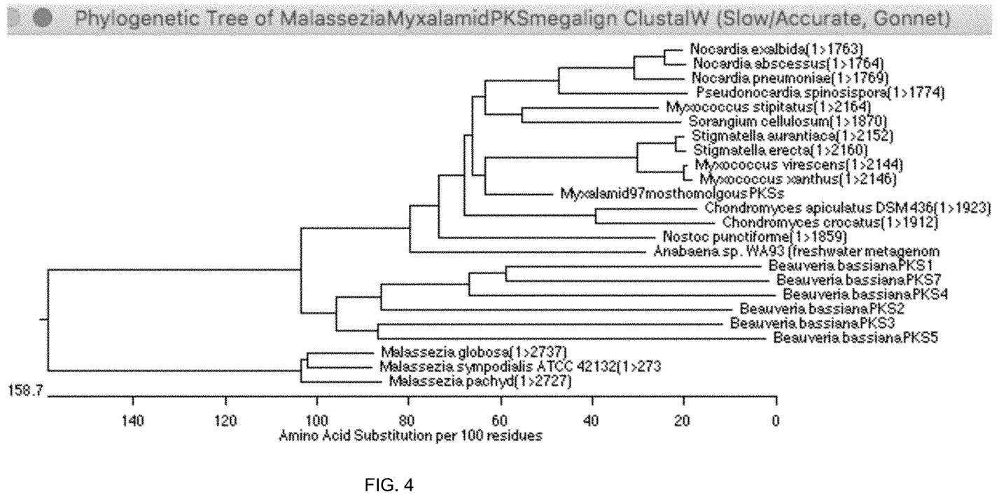

The inventors have discovered that entomopathogenic fungus Beauveria bassiana encodes polyketide synthetase enzymes that share homology with polyketide synthetase enzymes encoded in Malassezia globosa, Malassezia pachyderma (FIG. 3) and Malassezia sympodialis genomes. Beauveria bassiana is known to infect insects and produces mycotoxins that enter the central nervous system of the insects to cause symptoms similar to Parkinson's disease, namely involuntary muscle contractions, prostration with tremors, and eventually paralysis followed by death.

The inventors have discovered that chiropteratopathogenic fungus Pseudogymnoascus destructans encodes polyketide synthetase enzymes that share homology with polyketide synthetase enzymes encoded in the Malassezia globosa genome. Pseudogymnoascus destructans is known to infect bats and produces mycotoxins that enter the central nervous system of the bats to cause symptoms similar to Parkinson's disease.

The inventors have discovered that cyanobacteria Lyngbya majuscula genes encoding polyketide synthetase enzymes that produce the mycotoxin curacin A share homology with Malassezia genes encoding polyketide synthetase enzymes. Curacin A is known to interact with colchicine binding sites on tubulin, which inhibits microtubule polymerization, an essential process for cell division and proliferation of microtubules. Curacin A has also been characterized as a potent antiproliferative cytotoxic compound with known anti-neoplastic activity for several human cell lines.

Seborrhea and seborrheic dermatitis are common early symptoms of Parkinson's disease. Accordingly, the Malassezia responsible for the seborrhea and seborrheic dermatitis may cause the susceptibility, onset, development or progression in the Parkinson's disease state and symptoms. Mycotoxins produced by Malassezia are known to cause the redness and scaling symptoms of seborrheic dermatitis. It has been discovered through clinical observation coupled with deductive reasoning that increased seborrhea and worsening seborrheic dermatitis in patients with Parkinson's disease has a significant positive correlation with higher frequency of relapse or acceleration in progression of active Parkinson's disease. Thereby, detecting the mycotoxins produced by microorganisms responsible for the seborrhea and seborrheic dermatitis can be used to detect the susceptibility, onset, development or progression in the Parkinson's disease state and symptoms. Accordingly, the mycotoxins responsible for the seborrhea and seborrheic dermatitis may cause the susceptibility, onset, development or progression in the Parkinson's disease state and symptoms.

Intranasal Malassezia infection is a point of entry for Malassezial brain penetrant mycotoxins to access the central nervous system through the olfactory system. The presence of intranasal Malassezial infection correlates with olfactory dysfunction, such as decreased odor detection, identification, and discrimination, which is a common early symptom of Parkinson's disease.

### II. Therapeutic Methods

The present invention pertains, at least in part, to a method for treating a subject for Parkinson's disease by administering to the subject an effective amount of at least one compound or pharmaceutical composition of the invention. In one aspect, the compound(s) or pharmaceutical composition(s) may also be useful for treating a Malassezia infection. Accordingly, the present invention provides for simultaneously treating Parkinson's disease and Malassezia infection, which includes prophylactic measures to inhibit susceptibility and onset as well as curative measures to reduce or cure an ongoing or established Parkinson's disease or Malassezia infection.

The present invention provides a method for the prevention and/or treatment and/or mitigation of Parkinson's disease in a subject in need thereof including the step of administering to the subject therapeutically effective amount of a compound or pharmaceutical composition, comprising an anti-malassezial agent. Such a subject in need of a compound or pharmaceutical composition comprising an anti-malassezial agent may suffer from motor symptoms and dysfunctions that include one or more of bradykinesia, rest tremor (such as pill-rolling tremor), rigidity, postural instability, gait difficult (e.g., shuffling gait), hypomimia (masked facies), hypokinetic dysarthria, hypophonia, palilalia, dysphagia, sialorrhea, decreased spontaneous eye blink rate, eyelid opening apraxia, hypometric saccades, impaired vestibuloocular reflex, impaired upward gaze and convergence, respiratory distress, micrographia, incontinence, restless legs syndrome, sleep apnea, dystonia, myoclonus, forward-flexed posture, camptocormia (bent spine syndrome), Pisa syndrome, kyphosis, scoliosis, psychomotor retardation, freezing (motor block), and festination. Such patients in need of a compound or pharmaceutical composition comprising an anti-malassezial agent may also suffer from non-motor symptoms or dysfunctions that include one or more of cognitive and/or sensory dysfunction or impairment that include one or more of subcortical dementia, Lewy body dementia, psychomotor retardation, memory difficulty, learning and executive dysfunction (e.g., attention deficit disorder), language impairment, altered personality, impulse control dysfunction (e.g. obsessive behaviors), psychosis, hallucinations, delusions, depression, anxiety, social withdrawal, abulia, sleep disturbances (e.g., insomnia), fatigue, nausea, visual-spatial disturbances, blurred vision, visual contrast insensitivity, vision loss, autonomic dysfunction, olfactory dysfunction (e.g., anosmia), gastrointestinal dysfunction (e.g., constipation), sensory pain, sensory disturbances, dermatological dysfunction (e.g., seborrhea), seborrheic dermatitis (e.g., dandruff), and rhinorrhea.

As used herein the term “olfactory dysfunction” is a loss in the ability to smell or a change in perception of odors. Olfactory dysfunction can markedly impair the quality of life.

Olfactory dysfunction includes the conditions of anosmia, dysosmia, and hyposmia. Anosmia is the absence of the ability to smell and to detect odors. Hyposmia is a reduced ability to smell and to detect odors. Dysosmia is a disorder described as any qualitative alteration or distortion of the perception of smell. Qualitative alterations differ from quantitative alterations, which include anosmia and hyposmia. Dysosmia can be classified as either parosmia (also called troposmia) or phantosmia. Parosmia, also known as troposmia or cacosmia, is an olfactory dysfunction that is characterized by the inability of the brain to properly identify an odor's “natural” smell. Phantosmia, or phantom smell, also called an olfactory hallucination, is smelling an odor that is not actually there. Olfactory dysfunction can manifest as deficits in odor identification, discrimination, and detection. Olfactory dysfunction is very common in Parkinson's disease, with a prevalence of clinical diagnosis of olfactory dysfunction of about 70 to 90 percent in idiopathic Parkinson's disease. Olfactory dysfunction is at least as common among Parkinson's disease patients as resting tremor, a cardinal motor features of Parkinson's disease. The olfactory bulb in patients with Parkinson's disease typically reveals the presence of Lewy bodies in the neurons of the anterior olfactory nucleus and in mitral cells, with subsequent neuronal cell loss. Smell loss in Parkinson's disease may include deficits in all areas of olfaction: odor discrimination, odor identification and odor detection threshold. Impaired olfaction is associated with a significantly increased incidence of Parkinson disease in the elderly. Smell impairment in Parkinson's disease has been shown to appear early in the neurodegenerative process of the course of Parkinson's disease, often prior to the onset of motor symptoms.

In some embodiments, administration of the compound or pharmaceutical composition comprising an anti-malassezial agent treats one or more of the motor and/or non-motor symptoms of PD.

In some embodiments, subject improvement is measured by methods known in the art (e.g., ADAS-cog, MDRS, MoCA, CDR, FAB, D-KEFS VF, HVLT, TPCT, MMSE, QTCS, VMI, BTA, NPI, CSDD, BPRS, ADCS-ADL, QOL-AD, DAD, SE-ADL, PDQ-39, UPDRS, MDS-UPDRS, UPDRS-ADL, CGI, ADC-CGIC, CIBC-PLUS, and ZBI) and the response is statistically significant (e.g. test-retest reliability, Student's t-test, or the like).

In some embodiments, the improvement (e.g., amelioration or mitigation of Parkinson's disease and/or symptoms of Parkinson's disease) is measured with a subject's personal self-assessment and/or with the improvement measured by of the subject's caregiver or caregivers and/or with the improvement measured by the subject's healthcare provider or providers.

As used herein, the terms “subject” and “patient” are used interchangeably and include humans capable of suffering from, suffering from, or having symptoms of Parkinson's disease. For example, a subject can be an otherwise healthy individual who is at increased risk of acquiring Parkinson's disease because the person's genome contains a copy of the rs34637584(A) allele, a single nucleotide polymorphism (SNP) commonly referred to as the G2019S variant that is a well-known Parkinson's disease associated mutation rarely found in healthy, elderly people without Parkinson's disease and found with higher frequency in people with both the sporadic and familial types of Parkinson's disease. A person with a copy of the allele possesses a greatly increased chance of acquiring Parkinson's disease. “G2019S” refers to the change from glycine (encoded by rs34637584(G) allele) to serine (encoded by rs34637584(A) allele) at position 2019 of the leucine-rich repeat kinase 2 (LRRK2) protein (also known as dardarin), an enzyme that in humans is encoded by the PARK8 gene and is a member of the leucine-rich repeat kinase family of proteins.

Examples of other known Parkinson's disease associated mutations include variants rs112176450, rs35095275, rs34778348, rs3892097, rs287235, rs838552, rs283413, rs11868035, rs1057217, and rs6812193. In some embodiments, the invention provides methods of prophylactic treatment of Parkinson's disease by administering an effective amount of an anti-malassezial agent to a person with one or more mutations associated with increased risk of Parkinson's disease who has not acquired Parkinson's disease.

As used herein, the term “treat” or “treated” or “treating” or “treatment” refer to any type of action that imparts a modulating effect, which, for example, can be a beneficial effect, to a subject afflicted with a disorder, disease, or condition (e.g., Parkinson's disease), including improvement in the condition of the subject (e.g., in one or more symptoms), delay or reduction in the progression of the condition, and/or change in clinical parameters, disease or illness, etc., as would be well known in the art. The term “treatment” includes therapeutic and/or prophylactic treatment of Parkinson's disease, the diminishment or alleviation of at least one symptom associated with Parkinson's disease (e.g., bradykinesia, rest tremor, rigidity, insomnia, dementia, abulia, anosmia, and sialorrhea), and the eradication of one or more symptoms of Parkinson's disease.

In some embodiments, treatment can be a remission or cure of the condition of having Parkinson's disease. In one aspect, such treatment can provide simultaneous remission or cure for a Malassezial infection.

In some embodiments, treatment includes the arresting or slowing of progression of Parkinson's disease. In further embodiments, the stopping or slowing of the progression Parkinson's disease can be a decreased rate of increase in symptoms. The decreased rate of increase in symptoms can be measured, for example, by the Movement Disorder Society—Unified Parkinson's Disease Rating Scale (MDS-UPDRS) scores over time. The MDS-UPDRS is a commonly used clinical neurological rating scale that quantitatively measures longitudinal course of Parkinson's disease severity.

As used herein, “prevent” or “prevented” or “preventing” or “prevention” refer to prevention or delay of the onset of a disorder, disease, or condition (e.g., Parkinson's disease) and/or a decrease in the symptoms of Parkinson's disease in a subject relative to the symptoms of Parkinson's disease that would develop in the absence of the methods of the invention. The prevention can be complete, for example, the total absence of Parkinson's disease in a subject. The prevention can also be partial, such that the Parkinson's disease in a subject has reduced symptoms from that which would have occurred without the present invention. The terms “prevention”, “prophylactic treatment”, and “prophylaxis” may be used interchangeable and are intended to refer to prevention.

In some embodiments, the prophylactic treatment of Parkinson's disease in a subject comprises applying to the subject a composition comprising an anti-malassezial agent effective to prevent Malassezia fungi from infecting the subject, and/or to inhibit and/or kill a Malassezia fungi infection, so as to protect the subject from acquiring Parkinson's disease.

In some embodiments, treating Parkinson's disease may include a preceding step of identifying a subject as being infected, suspected of being infected, or at risk of infection, with Malassezia fungi.

In some embodiments, treating Parkinson's disease may include a preceding step of diagnosing a subject as being infected or at risk of infection, with a Malassezia fungi.

In some embodiments, treating Parkinson's disease may include a preceding step of identifying a site of a subject (e.g., the human nose cavity) as being colonized, suspected of being colonized, or at risk of colonization, with a Malassezia fungi. As used herein, “colonization” is the presence or establishment of a fungus organism or population of fungi at a particular site or location (e.g., the scalp) and/or the expansion of the numbers of the fungus organism by replication or recruitment of additional fungi.

As used herein, the term “infection” is encompassed by the term “colonization” and includes disease associated colonization and/or undesirable colonization.

In some embodiments the treatment, mitigation, or prevention for Parkinson's disease in a subject in need thereof comprises physical removal or reduction (e.g. debridement) of the Malassezia infection.

The physical removal of Malassezia at the location of infection can be carried out with any suitable surgical, mechanical or chemical means. In some embodiments, the means can be the use of a liquid, foam, gel, gel-solid, semi-solid compositions or gas applied at pressure to the location undergoing therapy. Following complete, partial, or attempted infection removal, the location may be contacted with an anti-malassezial agent. A composition used in the physical removal of the Malassezial infection or used as a wash solution before, during or after the removal may contain an anti-malassezial agent. Accordingly, some embodiments provide a debridement composition, for use in the treatments and methods of the invention. Such a debridement composition will typically be an aqueous sterile solution, and may additionally contain one or more abrasives (e.g., calcium carbonate, hydrated silica, hydrated aluminum oxide), detergents (e.g., nonoxynol-9, castile soap), and/or proteolytic enzymes (e.g. collagenase, elastase, pepsin, and trypsin).

In some embodiments, the debridement composition is an aqueous solution comprising a mixture of non-iodized sodium chloride and purified or filtered water warmed to about 98 degrees Fahrenheit (37 degrees Celsius). The aqueous solution may be hypertonic (e.g., 3% sodium chloride), isotonic (e.g., 0.9% sodium chloride) or hypotonic (e.g., 0.65% sodium chloride). The aqueous solution may have other components including, for example, pH modifiers, buffers (e.g., sodium bicarbonate), local anesthetic agents, preservatives, flavoring agents, coloring agents, agents that promote wound healing, agents that stop bleeding and/or promote clot formation, and other therapeutic and non-therapeutic components.

The term “administered,” “administering” or “administration” includes routes of administration which allow the anti-malassezial agents to perform their intended function(s) of preventing, mitigating, or treating Parkinson disease in a subject. Examples of routes of administration include parenteral (e.g., subcutaneous, intramuscular, intraorbital, intracapsular, intraspinal, intrasternal, intravenous, intradermal, intra-aurally, intraperitoneal, intraportal, intra-arterial, intrathecal, transmucosal, intra-articular, and intrapleural), transdermal, topical, epidural, and mucosal injection or infusion, as well as oral, intranasal, inhalation, insufflation, pulmonary, and rectal administration Examples of preferred routes of administration which may be used include injection, topical, oral, intranasal, intra-aural, subcutaneous, intravenous, inhalation and transdermal.

As used herein “co-administration” of one or more therapeutic agents means a treatment regimen wherein two or more active agents (e.g. a topical anti-malassezial agent, an oral anti-malassezial agent and an anti-Parkinson's agent) are administered to a patient by one or more administration routes (e.g. orally, topically, parenterally) simultaneously, in either separate or combined formulations, or sequentially at different times separated by minutes, hours or days, but in some way act together to provide the desired therapeutic response.

In some embodiments, the anti-malassezial agent is administered in combination with a pharmaceutically acceptable carrier. Examples of such carriers include those suitable for injection, topical, oral, intranasal, subcutaneous, intravenous, inhalation and/or transdermal administration.

Depending on the route of administration, an anti-malassezial agent may be coated with or contained within a material to protect it from natural conditions which may detrimentally affect the ability of the anti-malassezial agent to perform its intended function. For example, a sustained-release formulation may be designed to release the one or more anti-malassezial agents in a targeted part of the intestinal tract, mucosa (e.g., nasal passages), scalp, or respiratory tract, possibly over a period of time. The administration of the anti-malassezial agent is done at dosages and for periods of time effective to prevent, mitigate, or treat Parkinson's disease or to reduce or obviate the risk of Parkinson's disease. In some embodiments, the daily dosage of the anti-malassezial agent is about 50 mg, 100 mg, 150 mg, 200 mg, 250 mg, 300 mg, 350 mg, 400 mg, or higher. In a preferred embodiment the anti-malassezial agent is fluconazole and the daily dosage is 100 or 200 mg per day. In other embodiments, the anti-malassezial agent is administered daily for a period of at least four weeks, three months, at least four months, at least five months, at least six months, or longer.

In a more preferred embodiment, the anti-malassezial agent is fluconazole, the daily dosage is 100 mg or 200 mg per day, and the period of administration is at least 4 weeks, two months, or three months. In other embodiments, the anti-malassezial agent is given once a week for at least three, four, eight, twelve, or more weeks. In a preferred embodiment, the anti-malassezial agent is fluconazole and is administered at 300 mg once weekly for at least three, four or eight weeks. In a preferred embodiment the daily or weekly dosages of the anti-malassezial agent is administered orally. In a more preferred embodiment, fluconazole is administered orally at a daily dosage of 100 mg or 200 mg for at least four weeks, or at a weekly oral dosage of 300 mg for at least three, four or eight weeks.

Dosage regimes may be adjusted for purposes of improving the therapeutic or prophylactic response of the anti-malassezial agent. For example, several divided doses may be administered daily or the dose may be proportionally reduced as indicated by the exigencies of the therapeutic situation. In one embodiment, the first dose of the anti-malassezial agent given on day one of treatment is about 100 mg, 200 mg, 300 mg, 400 mg, 500 mg or higher, followed by a daily dose of about 50 mg, 100 mg, 150 mg, 200 mg, 250 mg, 300 mg, 350 mg, 400 mg, or higher for at least three months. In a preferred embodiment, the anti-malassezial agent is fluconazole, the starting dosage on day one of treatment is 400 mg given orally, the subsequent daily dosage is 200 mg per day given orally, and the period of administration is at least three months. Suitable pharmaceutical vehicles or dosage forms for injectable compositions, implants, and systemic administration are known.

For topical administration, percentages of anti-malassezial agent in a topical delivery vehicle may range from 0.01% to 10% (w/w). The anti-malassezial agent in a topical formulation preferably makes up from about 0.1% to about 5%, preferably from about 0.5% to about 3% of the total composition on a weight basis. Topical formulations include, but are not limited to, solutions, gels, fluid gels, emulsion gels and lotions.

The anti-malassezial agent may be administered intra-nasally and can be formulated into a variety of intranasal administrable compositions of the invention, such as washes, foams, gels, powders, and the like.

In some embodiments, the intranasal administration comprises irrigating the nasal passages with an anti-malassezial agent. In some embodiments, the anti-malassezial agent is administered to a patient by exposing the deeper reaches of the nasal cavity to effective amounts of an anti-malassezial agent, orally (e.g., gargling with an anti-malassezial agent formulated as a mouthwash), by inhalation (including nasal inhalation) and/or by nasal spray or drops.

Preferably, the anti-malassezial agent is administered topically to the “danger triangle” of the face (i.e., the area bounded by each lateral corner of the mouth (nasolabial fold) to the bridge of the nose, including the nose and maxilla) alone and/or exposing the deeper reaches of the nasal cavity by topically applying the anti-malassezial agent to the inner nasal surfaces and/or by intranasal administration. Preferably, olfactory dysfunction such as anosmia (i.e. the inability to perceive odor or a lack of functioning olfaction) is treated by topical administration of the anti-malassezial agent alone to the “danger triangle” of the face and/or by exposing the deeper reaches of the nasal cavity by topically applying the anti-malassezial agent to the inner nasal surfaces and/or by intranasal administration. Preferably olfactory dysfunction is treated by topical administration of an anti-malassezial agent alone. Preferably olfactory dysfunction is treated by topical administration of an anti-malassezial agent in combination with an oral anti-malassezial agent and/or in combination with an anti-Parkinson's agent.

The anti-malassezial agent may be administered in the intra-aurally (i.e., the ear canal) and can be formulated into a variety of intra-aural administrable compositions of the invention, such as washes, foams, gels, powders, and the like.

In some embodiments, the intra-aural administration comprises irrigating the ear canal with an anti-malassezial agent. In some embodiments, the anti-malassezial agent is administered to a patient by exposing the deeper reaches of the ear canal to effective amounts of an anti-malassezial agent, via swab stick application (e.g., Q-Tip containing an anti-malassezial agent formulated as a powder or gel), by ear wick (e.g., an ear wick impregnated with clotrimazole cream inserted into ear of patient by a physician in clinic and changed every third day for nine days), and/or by aural spray or drops. In some embodiments, the anti-malassezial agent is administered to a patient in conjunction with a drying agent. In some embodiments, the anti-malassezial agent is administered to a patient in conjunction with debridement of ear wax such as, for example, mechanical suctioning of the ear canal and/or application of Burow's solution or 5% aluminum acetate solution.

In some embodiments, some examples of drops for use in treating Malassezia present in the ear include clotrimazole: in a 1 percent solution) or flumetasone pivalate (e.g. in a 0.02 percent solution) plus clioquinol (e.g., in a 1 percent solution).

In some embodiments, an example of anti-malassezial cream for use in treating Malassezia present in the ear includes clotrimazole 1 percent cream. In some embodiments, the cream formulation suitable for treating Malassezia present in the ear is comprised of an anti-malassezial antifungal agent combined with benzyl alcohol, cetostearyl alcohol, cetyl esters wax, 2-octyldodecanol, polysorbate 60, purified water, and/or sorbitan monostearate.

In some embodiments, an anti-malassezial agent is administered to a patient by a liquid stream lavage (also referred to as a “neti pot”). In this approach, the anti-malassezial agent formulated as a solution is fed by gravity or slight pressure into one nostril and the composition exits the other nostril. An artificial pressure may also be used to feed the solution. An example of a device that creates artificial pressure to feed the solution is a pulsating irrigation device. An example of a pulsating irrigation device is the Grossan HydroPulse manufactured by Hydro Med, Inc. (Los Angeles, Calif.).

In some embodiments, a water vapor (mist) and/or other vapor based composition is administered into the nose and onto the nasal sinus and/or contiguous/adjacent nasal structures (e.g., olfactory fossa, olfactory bulb, and cribriform plate).

Some examples of descriptions of intranasal administration suitable for antifungal compounds and pharmaceutical formulations thereof are disclosed in U.S. Pat. No. 6,291,500 (to Ponikau) and U.S. Pat. No. 6,647,980 (to Gizurarson), which are incorporated herein by reference in their entirety.

The anti-malassezial agent may be administered topically to the skin and other body surfaces. and can be formulated into a variety of topically administrable compositions, such as lotions, solutions, suspensions, shampoos, foams, gels or ointments. Preferably, the anti-malassezial agent is administered topically in the absence of oral co-administration of an anti-malassezial agent. Preferably, the anti-malassezial agent is administered topically in combination with oral co-administration of an anti-malassezial agent.

In some embodiments, the anti-malassezial agent may be administered in combination with a different medication used to treat the symptoms of Parkinson's disease referred to herein as an “anti-Parkinson's disease medication or drug, or agent”. The anti-Parkinson's disease medication may be any medication known to the art to treat the symptoms of Parkinson's disease and is preferably selected from the group consisting of carbidopa-levodopa (e.g., Duopa), pramipexole (e.g., Mirapex), ropinirole (e.g., Requip) and rotigotine (e.g. Neupro), apomorphine (e.g., Apokyn), selegiline (e.g., Zelapar), rasagiline (e.g., Azilect), entacapone (e.g., Comtan), tolcapone, bromocriptine, benztropine (e.g., Cogentin), trihexyphenidyl, and amantadine. As used herein, a “different anti-Parkinson's disease medication” does not include an anti-malassezial agent of the invention.

In some embodiments, the co-administration of the anti-malassezial agent with another compound includes administration of the anti-malassezial agent first, followed by the compound and administration of the compound.

In some embodiments, the co-administration of the anti-malassezial agent with another compound includes administration of the compound first, followed by the anti-malassezial agent.

In some embodiments, the co-administration of the anti-malassezial agent with another compound includes administration of the anti-malassezial agent at the same time as the other compound.

When used herein, the term “therapeutically effective amount” or “effective amount” includes an amount of the therapeutic or treatment composition that provides a prophylactic or therapeutic benefit in the treatment, prevention, or management of a disease or a symptom of a disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The term “therapeutically effective amount” or “effective amount” of the anti-malassezial agent includes an amount of the anti-malassezial agent that is sufficient in treating, mitigating, or preventing Parkinson's disease. The term “effective amount” of the anti-malassezial agent also means an amount of the anti-malassezial agent to be administered necessary to treat or prevent a disease or disorder mediated by deficiency in nigral dopaminergic neurons. The term “effective amount” of the anti-malassezial agent also means the amount necessary to improve cognitive and/or motor function in a subject, or reduce a decline in cognitive and/or motor function. The term, “effective amount” of the anti-malassezial agent also includes the amount of the anti-malassezial agent required to prevent dopaminergic neuronal cell death in the substantia nigra. The term “effective amount” also includes the amount of the anti-malassezial agent with sufficient biostatic or biocidal activity against Malassezia infection to diminish or alleviate a symptom of Parkinson's disease.

A therapeutically effective amount can be readily determined on an individual basis and will be based, in part, on the severity of Malassezia infection and the activity of the specific anti-malassezial agent. Thus, a therapeutically effective amount of an anti-malassezial agent can be determined by one of ordinary skill in the art using no more than routine experimentation in clinical management of a subject. For example, the specific amount that is therapeutically effective may be readily determined by ordinary medical practitioner, and may vary depending on factors known in the art, such as, e.g. the type and location of infection, the patient's history (including genetic and medical history), sex, age, the patient's family history (including genetic and medical history), the patient's history of previous treatment modalities of Parkinson's disease and/or Malassezia infection, the patient's history of progression of Parkinson's disease, the stage of Parkinson's disease, genetic risk of Parkinson's disease, and the current administration of other therapeutic agents. Another factor influencing clinical management of a subject is the patient's side effect profile history. For example, previous gastro-intestinal (GI) upset and/or elevated liver enzyme levels (e.g., Alanine transaminase (ALT) and Aspartate transaminase (AST)) in relation to a patient's previous antifungal compound use may influence the determination of a specific treatment regimen of a subject in need of treatment, mitigation, or prevention of Parkinson's disease.

The dosage ranges for the administration of are those that produce the desired effect. Generally, the dosage will vary with the age, weight, condition, and sex of the patient, and the extent of disease. A person of ordinary skill in the art, given the teachings of the present specification, may readily determine suitable dosage ranges.

In some embodiments, a therapeutically effective amount of the anti-malassezial agent includes a precise dosage level determined by an attending physician or other health care provider and will depend upon well-known factors, including route of administration, and the age, body weight, sex, concomitant therapies, patient medical history including previous drug tolerances, general health of the patient; the nature, severity and clinical stage of Malassezia infection, and/or other clinical dosing factors known in the art.

### III. Anti-Malassezial Agents

As used herein, the term “anti-malassezial agent” is any substance that has a significant biocidal and/or biostatic activity against Malassezia fungi for use in the treatment, mitigation, or prevention of Parkinson's disease. In some embodiments, the anti-malassezial agent inhibits the growth of one or more Malassezia organisms in a subject receiving said agent. In some embodiments, the anti-malassezial agent kills one or more Malassezia organisms in a subject receiving said agent.

In some embodiments, the anti-malassezial agent is a substance that that has a biocidal and/or biostatic activity for Malassezia fungi. In further embodiments, the anti-malassezial agent is a substance that that has a biocidal and/or biostatic activity that is relatively specific and selective for Malassezia fungi.

In some embodiments, anti-malassezial comprise anti-fungal compounds that have biocidal and/or biostatic activity for Malassezia fungi.

Examples of anti-fungal compounds include polyene antifungal drugs (e.g. amphotericin B, natamycin, rimocidin, nystatin, candicidin, hamycin, filipin, perimycin), imidazole antifungals (e.g., miconazole, ketoconazole, climbazole, clotrimazole, econazole, enilconazole, omoconazole, bifonazole, butoconazole, fenticonazole, isoconazole, lanoconazole, luliconazole, oxiconazole, sertaconazole, sulconazole, tioconazole), triazole antifungals (e.g., fluconazole, fosfluconazole, itraconazole, isavuconazole, ravuconazole, posaconazole, voriconazole, terconazole, albaconazole), thiazole antifungals (e.g., abafungin), allylamine antifungals (e.g. terbinafine, naftifine, butenafine, amorolfine), echinocandin antifungals (e.g. anidulafungin, caspofungin, micafungin), hydroxypyridones (e.g., ciclopirox, piroctone olamine), tolnaftate, chlovalicin, ovalicin, fumagillin, sulfur, lithium gluconate, lithium succinate, zinc carbonate, polytar (e.g., coal tar), a thiocarbamate (e.g., tolnaftate), Whitfields ointment, and zinc pyrithione.

Preferred anti-fungal compounds include lipase inhibitors. Preferred lipase inhibitors include, but are not limited to, orlistat and cetilistat. Preferably, lipase inhibitors are topically administered to a patient to treat disease in accordance with the invention.

In some embodiments, amphotericin B is formulated as a liposomal amphotericin B, amphotericin B colloidal dispersion, amphotericin B lipid complex, deoxycholate amphotericin B, or amphotericin B oral suspension.

In some embodiments, the anti-malassezial agent may be an antifungal drug that is administered via systemic routes of administration (e.g., amphotericin B, ketoconazole, fluconazole, fosfluconazole, itraconazole, posaconazole, voriconazole, and terbinafine).

In some embodiments, the anti-malassezial agents will be an antifungal drug that is commonly administered as a non-systemic treatment (e.g., a topical treatment). Representative examples of such antifungal drugs include amphotericin B, natamycin, nystatin, candicin, hamycin, perimycin, miconazole, ketoconazole, clotrimazole, econazole, omoconazole, bifonazole, butoconazole, fenticonazole, isoconazole, oxiconazole, sertaconazole, sulconazole, tioconazole, fluconazole, fosfluconazole, isavuconazole, ravuconazole, terconazole, albaconazole, abafungin, terbinafine, naftifine, butenafine, amorolfine, tolnaftate, ciclopirox, zinc pyrithione; sulfur; salicylic acid, benzoic acid, coal tar (polytar), and tretinoic acid.

Examples of preferred anti-malassezial agents that are antifungal drugs, useful in the present invention, include ciclopirox olamine, zinc pyrithione, amphotericin B, nystatin, ketoconazole, miconazole, fluconazole, itraconazole, and voriconazole.

One preferred anti-malassezial agents for use in the invention is the tetrazole, (R)-2-(2,4-difluorophenyl)-1,1-difluoro-3-(1H-tetrazol-1-yl)-1-(5-(4-(2,2,2-trifluoroethoxy)phenyl)pyridin-2-yl)propan-2-ol (CAS#: 1340593-59-0). The generic name for this compound is oteseconazole. The research name for this compound is VT-1161.

One preferred compound is the leucyl-tRNA synthetase inhibitor (inhibits LeuRS), 5-fluorobenzo[c][1,2]oxaborol-1(3H)-ol. The generic name for this compound is tavaborole, its tradename is kerydin and its research name is AN-2690.

One preferred compound is the succinate dehydrogenase inhibitor. 2-(3,5-dimethyl-1H-pyrazol-1-yl)-5-methylphenol (CAS#: 1391758-52-3). The research name for this compound is ME-1111.

One preferred compound is the anti-fungal drug APX-001 (also called E1210). APX001 is a broad-spectrum antifungal drug candidate with a novel mechanism of action for the treatment of life-threatening, invasive fungal infections caused by Candida, Aspergillus and rare, hard-to-treat molds, and strains that are resistant to standard-of-care antifungal therapy. APX001 is in Phase 1 human clinical studies and is being developed by Amplyx Pharmaceuticals (San Diego, Calif.).

The antifungal drug may be used in any convenient form, including any pharmaceutically acceptable salt or hydrate. References to disclosed antifungal drugs extends to any analogs thereof, compounds which mimic their activity, and/or isomeric form in which the compound may exist as well as mixtures of two or more isomers, e.g. racemic mixtures. The invention contemplates compounds that inhibit fungal growth through an increase in copper at the location of the Malassezia infection.

In accordance with the invention, anti-inflammatory compounds and antiseptic compounds may also be considered to be anti-malassezial agents to the extent that these compounds have biocidal and/or biostatic activity for Malassezia fungi and to the extent that administration of these compounds is not deleterious to the subject, even though anti-inflammatory compounds and antiseptic compounds have a broad spectrum of biocidal and/or biostatic activity that do not display appreciable specificity or selectivity for Malassezia fungi over other cell types (e.g. bacteria, archaea, protists, plants, and animals).

In some embodiments, the antiseptic compound mitigates, treats, or prevents Parkinson's disease by decreasing or preventing Malassezia from coming in contact with the subject in need of mitigation, treatment, or prevention of Parkinson's disease.

In some embodiments, the antiseptic compound is formulated as a laundry composition comprising at least one antiseptic compound, the composition preferably being a laundry detergent or laundry conditioner. In some preferred embodiments, the antiseptic compound of the laundry composition comprises a bleach solution.

The varieties of anti-fungal compounds encompassed by the group of laundry compositions are well-known to those skilled in the art. For example, antifungal compounds for use in laundry compositions useful in the present invention and formulations thereof are disclosed in U.S. Pat. No. 7,987,539 (to Pestell et al.), U.S. Pat. No. 4,235,599 (to Davis et al.), and U.S. Pat. No. 6,228,127 (to Reinehr et al.), which are incorporated herein by reference in their entirety.

Anti-inflammatory compounds useful in the methods and compositions of the present invention include, but are not limited to, steroidal anti-inflammatory drugs and non-steroidal anti-inflammatory drugs (NSAIDs). Examples of preferred anti-inflammatory compounds, useful in the present invention, include ibuprofen and betamethasone.

In some embodiments, a safe and effective amount of an anti-inflammatory compounds may be added to a composition of the present invention, preferably from about 0.1% to about 25%, more preferably from about 0.5% to about 20%, of the composition. The exact amount of anti-inflammatory compound to be used in the methods and compositions will depend on the particular anti-inflammatory compounds utilized since such compounds vary widely in potency and toxicity.

Steroidal anti-inflammatory drugs, include corticosteroids such as, alpha-methyl dexamethasone, dexamethasone-phosphate, hydrocortisone, hydroxyltriamcinolone clobetasol valerate, desonide, desoxymethasone, diflorasone diacetate, diflucortolone valerate, fluadrenolone, flumethasone pivalate, fluclorolone desoxycorticosterone acetate, fluosinolone acetonide, fluocinonide, flucortine butylester, fluocortolone, paramethasone, prednisolone dexamethasone, dichlorisone, acetonide, fludrocortisone, prednisolone acetate, loteprednol etabonate, medrysone, amcinafel, amcinafide, betamethasone, chloroprednisone, chloroprednisone acetate, clocortelone, prednisone, beclomethasone dipropionate, budesonide, flurandrenolone, halcinonide, dichlorisone, difluprednate hydrocortisone acetate, hydrocortisone butyrate, methylprednisolone, triamcinolone acetonide, cortisone, fludrocortisone, difluorosone diacetate, fluradrenolone acetonide, clescinolone, flucloronide, flunisolide, fluoromethalone, fluperolone, fluprednisolone, hydrocortisone valerate, hydrocortisone cyclopentylpropionate, hydrocortamate, meprednisone, fluticasone propionate, rimexolone, and mometasone furoate.

A second class of anti-inflammatory compounds which is useful includes the non-steroidal anti-inflammatory drugs (NSAIDs). Suitable NSAIDs include, but are not limited to salicylates (e.g., acetylsalicylic acid, amoxiprin, methyl salicylate, choline salicylate, magnesium salicylate, and salicylamide), arylalkanoic acids (e.g., diclofenac), profens (e.g., ibuprofen, alminoprofen, benoxaprofen, carprofen, and naproxen), N-arylanthranilic acids, pyrazolidine derivatives (e.g., phenylbutazone), oxicams, cyclooxygenase-2 inhibitors (e.g., celecoxib) and sulphonanilides (e.g., nimesulide).

A third class of anti-inflammatory compounds which is useful includes the topical calcineurin inhibitors. Suitable topical calcineurin inhibitors include, but are not limited to tacrolimus and pimecrolimus.

The varieties of compounds encompassed by the group of anti-inflammatory compounds are well-known to those skilled in the art. Mixtures and admixtures of anti-inflammatory compounds may also be employed, as well as the pharmaceutically acceptable salts and esters of these compounds.

Finally, so-called “naturopathic” anti-malassezial agents are useful in the methods and compositions of the present invention. Examples of naturopathic compounds, useful in the present invention, include but are not limited to cinnamics (e.g. cinnamon), vanilla (e.g. vanillin), tea tree oil, citronella, camphor, coconut (e.g., SEBCO), antioxidants (e.g., vitamin D), turmeric, cypress extracts, lavender, colloidal silver, colloidal copper, colloidal zinc, limonene, lemon myrtle, lemongrass, neem seed, olive leaf extract, orange oil, palmarosa oil, patchouli oil, beeswax, honey, aloe vera, yoghurt-acidophilus-milk (YAM), acidophilus, yoghurt, phenylalanine, methylated spirits, seaweed, Allium cepa, and Allium sativum (e.g. allicin).

The varieties of compounds encompassed by the group of naturopathic agents are known to those skilled in the art. Mixtures and admixtures of naturopathic agents may also be employed, as well as the pharmaceutically acceptable derivatives, extracts, oils, salts and esters of these agents.

The present invention encompasses the use of a single anti-malassezial agent or a plurality (e.g., mixture) of different anti-malassezial agents. Thus, for example, one anti-malassezial agent or a combination of different anti-malassezial agents (e.g. two or more) may be used.

### IV. Anti-Malassezial Phototherapy

In some embodiments, the present invention provides anti-malassezial phototherapeutics to mitigate, treat, or prevent Parkinson's disease in a subject in need thereof comprising irradiating a location with a Malassezia infection with sufficient electromagnetic radiation to inhibit the growth of or kill the Malassezia, wherein the radiation is administered from a minimum wavelength of about 350 nanometers (nm) to a maximum wavelength of about 450 nm. Preferably the radiation is a wavelength of about 375, 380, 385, 390, 395, or 400 nm. More preferably the radiation is a wavelength of about 378, 379, 380, 382, 383, 384, 385, 386, 387, 388, 389, 390, 391, 392, 393, 394, 395 nm. Even more preferably still the radiation is a wavelength of about 391.5 391.6, 391.7, 391.8, 391.9, 392, 392.1, 492.2, 392.3, 392.4, 392.5, 392.6, 392.7, 392.8, 392.9, 393, 393.1, 393.2, 393.3, 393.4, or 393.5 nm.

As used herein the term “anti-malassezial phototherapy”, “anti-malassezial phototherapeutics”, or “UV light therapy” refers to an artificial source of light of the frequencies of the present invention for use in mitigation, treatment, or prevention of Parkinson's disease. The frequency or frequencies of a given light source of the present invention can be across the entire range, a portion of the range or can be limited to a single frequency or band of frequencies within the range.

Examples of anti-malassezial phototherapy useful in the present invention include ultraviolet light therapy (phototherapy) comprising PUVA (psoralens plus ultraviolet A radiation), broadband UVA, broadband UVB, combined UVA and UVB, narrow-band UVB, or UVA1. In some embodiments, the phototherapy is administered as a medium dose of UVA1 in combination with a medium dose of narrow-band UVB. In a preferred embodiment, the phototherapy is administered as a medium dose of UVA1 in combination with a medium dose of narrow-band UVB administered within the range of 30 to 60 J/cm2 (joules divided by centimeters squared).

In some embodiments, anti-malassezial phototherapy is a photo/antifungal combination therapy that is administered in combination with an anti-malassezial agent. In a preferred embodiment of the photo/antifungal combination therapy, phototherapy is administered in combination with a topical anti-fungal compound such as selenium sulfide, ketoconazole, or miconazole. Topical emollients (e.g., propylene glycol) may be useful in phototherapy since phototherapy may increase skin dryness.

In some embodiments, a location is treated with separate light sources simultaneously. In some embodiments, a single dose exposure to light is from about 1 to 30 minutes. More preferably the exposure is from 6 to 15 minutes. Most preferably the exposure is 10 to 12 minutes. In some embodiments, the subject is administered a dose exposure 2 to 5 times daily, once a day, 1 to 3 times weekly, or monthly. In a preferred embodiment, the phototherapy is administered between four to six times per week. In some embodiments, the light source may be positioned very close or in contact with the body of the subject. In some embodiments, the light source is one that does not generate large amounts of heat when in use. In some embodiments, the light source is a light emitting diode (LED). In some embodiments, the light source is an LED light bulb having a light output of about 0.7 lumens at between 3 and 4 volts. In some embodiments, the light source is generated by a device that is hands-free, battery powered, and/or portable.

In some embodiments, the light source is encompassed in a tanning lamp or bed well known in the art. In some embodiments, the anti-malassezial phototherapy mitigates, treats, or prevents Parkinson's disease by preventing Malassezia from coming in contact with a subject in need of mitigation, treatment, or prevention of Parkinson's disease. Descriptions of phototherapy suitable for use in preventing Malassezia from coming in contact with the subject are disclosed in U.S. Pat. No. 5,664,340 (to Brown) and U.S. Pat. No. 8,109,981 (to Gertner et al.), which are incorporated herein by reference in their entirety.

### V. Pharmaceutical Compositions

The present invention pertains, at least in part, to compositions for treating Parkinson's disease in a subject comprising an effective amount of an anti-malassezial agent or salt thereof and a pharmaceutically acceptable carrier.

Compounds disclosed herein may be formulated with one or more pharmaceutically acceptable excipients, diluents, carriers, etc. to produce pharmaceutical compositions for administration to a subject, by any of various pharmaceutically approved routes, for the prevention, treatment or mitigation of Parkinson's disease.

Pharmaceutical compositions of the present invention may be made into a wide variety of product types. Product types include, but are not limited to solutions, lotions, creams, beach products, gels, hydrogels, sticks, sprays, nebulizers, pads, plasters, ointments, pastes, mousses, foams, and cosmetics. These product types may comprise several types of carrier systems including, but not limited to solutions, emulsions, gels, foams, and solids. The topical pharmaceutical compositions of the present invention formulated as solutions typically include a pharmaceutically-acceptable aqueous or organic solvent.

Pharmaceutical compositions of the present invention may be prepared as a concentrate form, so that consumer products can be produced therefrom by simple dilution, without necessarily blending in of other formulating components. In some embodiments, the concentrate form is an article of commerce.

The pharmaceutical compositions of the present invention may be suitable for any route of administration which allows an anti-malassezial agent to perform the intended function of the anti-malassezial agent; namely preventing, mitigating, or treating Parkinson's disease in a subject in need thereof. Non-limiting examples of routes of administration of pharmaceutical compositions of the present invention include topical, intranasal, systemic, or oral administration.

In some embodiments, the pharmaceutical composition comprises an anti-malassezial agent and a pharmaceutically acceptable carrier for use in the treatment, prevention, or mitigation of Parkinson's disease.

In some embodiments, the pharmaceutical composition comprises an anti-malassezial agent and a pharmaceutically acceptable carrier for use in the treatment, prevention, or mitigation of fatigue, particularly mental fatigue, associated with Parkinson's disease.

The pharmaceutical compositions of the present invention further comprise packaged pharmaceutical compositions to treat, mitigate, or prevent Parkinson's disease. A packaged pharmaceutical composition (also referred to as a “kit”) is intended to include at least one anti-malassezial agent, packaged with instructions (e.g., instructions for once a day administration, twice a day administration, etc.) for administering the anti-malassezial agent(s) as a treatment, mitigation, or prevention of Parkinson's disease. The package can contain one or more containers filled with one or more of the ingredients of the pharmaceutical compositions of the invention.

In some embodiments, the packaged pharmaceutical compositions include one or more containers holding one or more anti-malassezial agents, alone or in combination with one or more pharmaceutically acceptable carriers, along with instructions for use for the treatment, mitigation, and/or prevention of Parkinson's disease.

In some embodiments, the packaged pharmaceutical composition may also include one or more medications known in the art to treat the symptoms of Parkinson's disease.

In some embodiments, an anti-malassezial agent of the packaged pharmaceutical composition is a member of one of the preferred antifungal compounds described herein.

In some embodiments, the packaged pharmaceutical compositions include a notice in a form prescribed by a governmental agency regulating the manufacture, use or sale of a pharmaceutical product or products, which notice reflects approval by the agency of manufacture, use or sale for human administration of a pharmaceutical composition of the present invention.

In some embodiments, the packaged pharmaceutical compositions include package labeling identifying an anti-malassezial agent along with instructions for use of the anti-malassezial agent for the treatment, mitigation, and/or prevention of Parkinson's disease.

In some embodiments, a pharmaceutical composition and/or packaged pharmaceutical composition of the present invention is sterilized.

The phrase “pharmaceutically acceptable” refers to those compounds, dosage forms, materials, and/or compositions which are, within the scope of sound medical judgment, suitable for use in contact with the tissues of human beings without excessive toxicity, irritation, allergic response, or other problem or complication, commensurate with a reasonable benefit/risk ratio.

The phrase “pharmaceutically acceptable carrier” includes a pharmaceutically acceptable material, composition or vehicle, such as a liquid or solid filler, diluent, excipient, solvent or encapsulating material, involved in carrying or transporting the anti-malassezial agent within or to the subject such that the anti-malassezial agent can perform the anti-malassezial agent's intended function, e.g. to treat Parkinson's disease. Each carrier must be “acceptable” in the sense of being compatible with the other ingredients of the formulation and not injurious to the subject.

Pharmaceutically acceptable carriers that may be useful in formulating pharmaceutical compositions of the present invention include but are not limited to lubricants, preservatives, stabilizers, solubilizers, penetrants, wetting agents, drying agents, bulking agents, fillers, emulsifiers, salts for influencing osmotic pressure, tonicity contributors (e.g., dextrose, mannitol, glycine and sodium chloride), buffers, antioxidants, colorings, flavorings and/or aromatic substances and the like which do not deleteriously react with the active compounds of the invention or each other.

Clearly, the skilled person can use other pharmaceutical formulations of the present invention containing anti-fungal compounds and pharmaceutically acceptable carriers. Examples of pharmaceutically acceptable carriers known in the art that may be useful in formulating pharmaceutical compositions of the present invention, and methods of forming such compositions, are described in detail by reference to standard textbooks such as Remington: The Science and Practice of Pharmacy, Twenty-Second Edition (Lippincott Williams & Wilkins, 2012); Handbook of Pharmaceutical Excipients, Seventh Edition (Pharmaceutical Press, 2012); Ansel's Pharmaceutical Dosage Forms and Drug Delivery Systems, Tenth Edition (Lippincott Williams & Wilkins, 2013); Modern Pharmaceutics, Fifth Edition (CRC Press, 2009); and in Harry's Cosmeticology, Ninth Edition (Chemical Publishing Company, 2015). Said standard textbooks are incorporated herein by reference in their entirety.

Examples of suitable pharmaceutically acceptable carriers include water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, and the like.

In some embodiments, the pharmaceutical composition comprising an anti-malassezial agent and a pharmaceutically acceptable carrier has a pH of greater than 4.0.

In some embodiments, the pharmaceutical composition of the invention comprises an anti-malassezial agent, a pharmaceutically acceptable carrier, and a medication to treat the symptoms of Parkinson's disease.

When used herein, the term “topical” includes references to formulations that are adapted for application to a body surface or body surfaces (e.g. the skin and mucous membranes). Mucous membranes that may be mentioned in this respect include the mucosa of the vagina, the penis, the urethra, the bladder, the anus, the mouth (including the mucosa of the cheek, the soft palate, the under surface of tongue, the ceiling and floor of the mouth), the nose (including the nasal cavity, olfactory fossa, and cribiform plate), the throat (including the mucosa of the pharynx, the larynx, the trachea and the esophagus), the bronchi, the lungs, the eye (including the lacrimal duct and conjunctiva), the ear (including the inner ear canal), and the like. Skin includes groin region (e.g. external genitals, anogenital area), scalp, scalp margin, neck, neckline, eyelids, eyebrow region, eyelashes, auricles, hairy areas of the head (e.g., eyelashes, eyebrows, mustache, beard), forehead, chin, cheeks, nail-beds (including fingers and toes), nasolabial folds, external ear canals, auricular areas, post-auricular creases, shoulders, elbows, back, sternal area, supra-sternal notch, supra-mammary area, mammary area (including areolar area), infra-mammary area, axillae, navel, and the like. Body surfaces include areas that are defined based on standard clinical/medical descriptions known in the art. For example, the so-called “danger triangle” of the face (i.e., the area bounded by each lateral corner of the mouth to the bridge of the nose, including the nose and maxilla) is considered a body surface.

When used herein, the term “topical administration” includes methods of delivery to a body surface or body surfaces (e.g. the skin or mucous membranes). Topical administration involves any form of administration which involves a body surface or body surfaces (e.g. the skin or mucous membranes). Topical administration includes laying on or spreading a compound of the present invention on the skin (e.g. the scalp) of a subject.

One of ordinary skill may readily determine the optimum amount of the topical formulation to be administered, administration methodologies and repetition rates. In general, it is contemplated that the topical formulations of the present invention will be applied in the range of once or twice or three or four or five times weekly up to once or twice or three or four or five times daily.

The topical formulation for use in the present invention may be in any form suitable for application to the body surface, such as a cream, lotion, sprays, solution, gel, hydrogel, foam, powder, ointment, paste, plaster, paint, shampoos, bio-adhesive, suspensions, aerosols, solids (e.g., bar soaps, deodorant), cosmetic formulations, and skin cleansing formulations, or the like, and/or may be prepared so as to contain liposomes, micelles, and/or microspheres. Such a formulation may be used in combination with an occlusive over-layer. In some embodiments, the occlusive over-layer allows moisture evaporating from the body surface to be maintained within the formulation upon application to the body surface and thereafter. In some embodiments, the occlusive over-layer provides the skin or mucosal membrane of the subject with a protective barrier from the eternal environment.

Topical formulations include any formulation suitable for topical delivery of a composition of the invention. Topical formulations may include those in which an active compound of the present invention is dissolved or dispersed in a dermatological vehicle known in the art (e.g. aqueous or non-aqueous gels, ointments, water-in-oil and oil-in-water emulsions). For example, constituents of such vehicles may comprise water, aqueous buffer solutions, non-aqueous solvents (e.g., ethanol, isopropanol, benzyl alcohol, propylene glycol, propylene glycol monolaurate, glycofurol and glycerol), oils (e.g. mineral oil, triglyceride, and dimethicone), a solubilising agent or solvent (e.g. a beta-cyclodextrin, such as hydroxypropyl beta-cyclodextrin, an alcohol, a polyol (such as ethanol, propylene glycol or glycerol); a thickening agent (e.g. hydroxyethylcellulose); a gelling agent (e.g. a polyoxyethylene-polyoxypropylene copolymer); a preservative (e.g. benzyl alcohol, benzalkonium chloride, and chlorhexidine); and a pH buffering agent (e.g. such as a mixture of dihydrogen phosphate and hydrogen phosphate salts).

The terms “pharmaceutically-acceptable aqueous solvent” and “pharmaceutically-acceptable organic solvent” refer to a solvent, which is capable of having dispersed (or dissolved therein) the active compound, and possesses acceptable safety properties (e.g., desired irritation and sensitization characteristics). Water is a typical aqueous solvent. Non-limiting examples of suitable organic solvents include: propylene glycol, butylene glycol, glycerol, butanediol, and mixtures thereof. Preferably, these solutions contain from about 0.01% to about 50% of the active compound, more preferably from about 0.1% to about 20%, and, for example, between about 0.1% and 10% of the active compound; and from about 1% to about 80% of an acceptable aqueous or organic solvent, more preferably from about 1% to about 30%.

In some embodiments, the topical pharmaceutical compositions of the present invention are formulated as an aerosol for administration to the subject as a spray. In further embodiments, a propellant is added to the solution composition of the aerosol to improve the desired activity of the aerosol.

Topical pharmaceutical compositions of the present invention may be formulated as a solution comprising an emollient. As used herein, “emollients” refer to materials used for the prevention or relief of dryness, as well as for the protection of the skin. Preferably, such compositions contain from about 0.1% to about 50% of the active compound and from about 2% to about 50% of a topical pharmaceutically acceptable emollient.

A lotion can be made from a solution carrier system. Lotions preferably comprise from about 0.1% to about 20%, more preferably from about 1% to about 5%, of the active compound; from about 1% to about 20%, preferably from about 5% to about 10%, of an emollient; and from about 50% to about 90%, preferably from about 60% to about 80%, water.

Another type of product that may be formulated from a solution carrier system is a cream. A cream of the present invention would preferably comprise from about 0.1% to about 20%, more preferably from about 1% to about 5%, of the active compound; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble. Typically, an ointment may also comprise from about 2% to about 10% of an emollient plus from about 0.1% to about 2% of a thickening agent.

If the carrier is formulated as an emulsion, from about 1% to about 10%, preferably from about 2% to about 5%, of the carrier system comprises an emulsifier. Emulsifiers may be nonionic, anionic or cationic.

Lotions and creams can be formulated as emulsions as well as solutions. Preferably such lotions comprise from about 0.1% to about 20%, more preferably from about 1% to about 5%, of the active compound; from about 1% to about 20%, preferably from about 5% to about 10%, of an emollient; from about 25% to about 75%, preferably from about 45% to about 95%, water; and from about 0.1% to about 10%, preferably from about 0.5% to about 5%, of an emulsifier. Such creams would preferably comprise from about 0.1% to about 20%, more preferably from about 1% to about 5%, of the active compound; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier. Single emulsion topical preparations, such as lotions and creams, of the oil-in-water type and water-in-oil type are known in the cosmetic art and are useful in the present invention. Micro-emulsion carrier systems are also useful in the present invention. These carrier systems are preferably combined with from about 1% to about 10% of the active compound. A method of formulating a gel or a cosmetic stick is by adding a suitable amount of a thickening agent to a cream or lotion formulation.

Various water-soluble materials may also be present in the compositions of this invention. These include humectants, proteins and polypeptides, preservatives and an alkaline agent. In addition, the topical compositions herein can contain conventional cosmetic adjuvants, such as dyes, opacifiers (e.g., titanium dioxide), pigments, and fragrances. Formulations of the invention suitable for oral administration may be in the form of capsules, pills, wafers, tablets, lozenges, cachets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the present invention may also be administered as an electuary, bolus, or paste.

In solid dosage forms of the invention for oral administration (capsules, tablets, wafers, pills, dragees, powders, granules and the like), the active ingredient is mixed with one or more pharmaceutically acceptable carriers (e.g., sodium citrate, dicalcium phosphate), and/or any of the following: fillers or extenders, such as starches, lactose, sucrose, glucose, mannitol, and/or silicic acid; binders, (e.g., carboxymethylcellulose, gelatin, sucrose and acacia); humectants (e.g. glycerol); disintegrating agents (e.g. agar-agar, calcium carbonate, tapioca starch); solution-retarding agents (e.g. paraffin); absorption accelerators; wetting agents (e.g. cetyl alcohol); absorbents (e.g. kaolin); lubricants (e.g., talc, magnesium stearate, solid polyethylene glycols, sodium lauryl sulfate); and coloring agents). In the case of capsules, tablets and pills, the pharmaceutical compositions may also comprise suitable buffering agents. Solid compositions of a similar type may also be employed as fillers in soft and hard-filled gelatin or gelatin-type capsules (e.g., employing such excipients as lactose, high molecular weight polyethylene glycols, and the like).

A tablet may be made by compression or molding, optionally with one or more accessory ingredients. Compressed tablets may be prepared using binder, lubricant, inert diluent, preservative, disintegrant, and/or surface-active or dispersing agent. Molded tablets may be made, for example, by molding in a suitable machine a mixture of the powdered compound moistened with an inert liquid diluent.

The tablets, and other solid dosage forms of the pharmaceutical compositions of the invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the pharmaceutical compositions of the invention therein using, for example, hydroxypropylmethyl cellulose in varying proportions to provide the desired release profile, other polymer matrices, liposomes and/or microspheres. The solid dosage forms may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the pharmaceutical compositions of the invention only, or preferentially, in a certain portion of the gastrointestinal tract, optionally, in a delayed manner. Examples of embedding compositions suitable for use include polymeric substances and waxes. The active compound may also be formulated in a micro-encapsulated form.

Liquid dosage forms for oral administration of an active compound include pharmaceutically acceptable emulsions, micro-emulsions, solutions, suspensions, syrups and elixirs. In addition to an active compound, the liquid dosage forms may contain inert diluents commonly used in the art, such as, for example, water or other solvents, solubilizing agents and emulsifiers (e.g., ethyl alcohol, isopropyl alcohol, ethyl carbonate, ethyl acetate, benzyl alcohol, benzyl benzoate, propylene glycol, glycerol, tetrahydrofuryl alcohol, polyethylene glycols and fatty acid esters of sorbitan, and mixtures thereof). Besides inert diluents, oral, intranasal, or ophthalmic compositions can also include adjuvants such as wetting agents, emulsifying and suspending agents, sweetening, flavoring, coloring, perfuming and preservative agents.

A wide variety of antifungal pharmaceutical formulations suitable for use in the present inventions are well known in the art. For example, descriptions of a variety of antifungal pharmaceutical formulations known in the art are found in: U.S. Pat. No. 7,101,889 (to Kaczvinsky, Jr. et al.), U.S. Pat No. 6,897,033 (to Dawson et al.) U.S. Pat. No. 6,656,928 (to McAdden), U.S. Pat. No. 6,046,176 (to Segal et al.), U.S. Pat. No. 6,558,710 (to Godfrey), U.S. Pat. No. 7,820,720 (to Cevc et al.), U.S. Pat. No. 4,006,222 (to Metzger), U.S. Pat. No. 7,732,450 (to Whitfield et al.), U.S. Pat No. 5,536,742 (to Mason), U.S. Pat. No. 5,730,965 (to Rapaport), U.S. Pat. No. 4,950,477 (to Schmitt), U.S. patent application Ser. No. 11/671,416 (Lawyer et al.), U.S. Pat. No. 7,193,084 (to Werling et al), U.S. patent application Ser. No. 12/446,166 (Schmaus et al.), U.S. patent application Ser. No. 14/004,344 (O'Neil, et al.), U.S. patent application Ser. 12/663,766 (Hernandez et al.), and U.S. Pat. No. 4,902,789 (to Michel, et al.), which are incorporated herein by reference in their entirety.

Examples of commercially available antifungal pharmaceutical formulations suitable for use in the present invention include: NIZORAL (ketoconazole 2% cream or shampoo), KENOZOLE-B (ketoconazole 2% & Beclomethasone 0.025% cream), MONISTAT (miconazole nitrate 2% cream), LAMISIL (terbinafine 1% solution or cream), ABELCET (amphotericin B 5 mg/mL lipid complex injection), LITHIODERM (lithium gluconate 8% gel), EFALITH (lithium succinate 8%/zinc sulfate 0.05% cream), LOTRIMIN (clotrimazole 1% cream, lotion, or solution), NOXAFIL (posaconazole 40 mg/mL oral suspension) and LOPROX (ciclopirox olamine 1% shampoo, gel, or cream), CANESTEN (clotrimazole 1% cream), HEAD & SHOULDERS (Zinc Pyrithione 1% shampoo), SELSUN BLUE (Selenium sulfide 1% shampoo).

In some embodiments, amphotericin B for the treatment, mitigation, or prevention of Parkinson's disease is formulated as amphotericin B for injection which is a sterile, nonpyrogenic, lyophilized cake (which may partially reduce to powder following manufacture) providing 50 mg amphotericin B and 41 mg sodium desoxycholate buffered with 20.2 mg sodium phosphates (consisting of mono and dibasic sodium phosphate, phosphoric acid and sodium hydroxide). Crystalline amphotericin B is insoluble in water; therefore, the active compound is solubilized by the addition of sodium desoxycholate to form a mixture which provides a colloidal dispersion for intravenous infusion following reconstitution. At the time of manufacture the air in the vial is replaced by nitrogen as a preservative.

The following examples and figures herein are disclosed for illustrative purposes only, to provide non-limiting illustrations and details of certain aspects of the present invention and are not intended to limit the scope of the invention.

### VI. Therapeutic Kits

The present technology can include kits that can be used for preventing, inhibiting, treating, or otherwise providing a therapy for inhibiting Parkinson's disease. The kit may be useful for subjects diagnosed as being susceptible to or having Parkinson's disease. The kits can include components described herein and/or in the claims.

In some embodiments, a kit can include an absorbent medium having an anti-malassezial agent. In some further embodiments, a kit can include a shower glove having the anti-malassezial agent in an absorbent region. In some yet further embodiments, the absorbent medium can be a sponge having the anti-malassezial agent.

In another aspect, the kit can provide articles of commerce and methods of marketing hair care compositions that can be used for a therapy for Parkinson's disease. In some embodiments, the article of commerce comprises: (1) a container; (2) a skin care composition contained within said container, wherein said skin care composition comprises an anti-malassezial agent; and, optionally, (3) a communication, wherein said communication communicates that use of said skin care composition can reduce the chance of becoming susceptible to Parkinson's disease or of developing Parkinson's disease. The communication can provide information of the link between a malassezial infection and becoming susceptible to Parkinson's disease or becoming diagnosed with Parkinson's disease.

In another aspect, the present invention provides methods of marketing skin care compositions that can be used to reduce the chance of becoming susceptible to Parkinson's disease or of developing Parkinson's disease by inhibiting, preventing, or treating a malassezial infection.

In some embodiments, the method comprises: (a) offering for sale a skin care composition comprising an anti-malassezial agent; (b) communicating that said composition can be used to reduce the chance of becoming susceptible to Parkinson's disease or of developing Parkinson's disease by inhibiting, preventing, or treating a malassezial infection.

In some embodiments, the skin care composition contains an antifungal for treating a malassezial infection for the purposes of inhibiting, preventing, or treating someone susceptible to or having Parkinson's disease and dandruff.

Any container from which the skin care composition can be stored and/or contained can be used herein. Suitable containers can include, but are not limited to, bottles, tottles, tubes, pouches, blister packs, boxes, tubs, jars, and cans. Furthermore, containers can include primary containers, which contain the hair care composition itself, or secondary containers, which contain at least one primary container that contains the composition. As used herein, “set of graphics” or “graphics” refers to the text and/or pictorial images that are disposed on a container. As used herein, “disposed on” means integral with and/or located on the container and can include, but is not limited to, disposed directly thereon (e.g., printed directly on the container), disposed indirectly thereon (e.g., printed on a sticker that is affixed to the outer portion of the container), and/or applied to the container by any other suitable means (e.g., sprayed, bonded, drawn, painted, printed, or molded). As used herein, “communication” means a message, and can include but is not limited to a printed (e.g., printed material attached directly or indirectly to the container), electronic, or broadcast message.

In one embodiment, the marketing described herein for a skin care product can be applied to any product that includes an anti-malassezial agent for treating a malassezial infection for the purpose of inhibiting, preventing, or treating someone susceptible to Parkinson's disease or someone having Parkinson's disease. Such products may be nasal products, hair products, ear products, cleaning products for nasal, skin, hair, ear, or other location, or the like. Additional information can be found in US 20120258185 and/or US20080059313, which both are incorporated herein by specific reference in their entirety.

### EXAMPLES

### Example 1

A 55-year old female patient visits her physician complaining seborrheic dermatitis of the scalp, neck, shoulders, and face. Patient suffers from Parkinson's disease that was diagnosed about 7 months ago presenting with initial presenting symptoms of anosmia, tremor, and bradykinesia. Patient is administered oral fluconazole 100 mg daily, topical clotrimazole 1% cream applied to the location of the seborrheic dermatitis twice daily, and tretinoin 0.04% cream applied to the location daily. This treatment regimen is continued for three months. At this time, patient examination demonstrates the seborrheic dermatitis infection is completely eradicated. At a 3-month follow-up from successful treatment of seborrheic dermatitis infection patient reports anosmia, has resolved. In addition, the Parkinson's disease symptoms of tremor and bradykinesia have decreased significantly.

### Example 2

A 30-year old male subject is determined by genetic testing to possess LRRK2 gene mutation G2019S which is associated with an increased risk of Parkinson's disease. Subject is administered zinc pyrithione formulated as a shampoo once daily. The zinc pyrithione shampoo is applied to the entire surface of the body excluding mucosal membranes. The shampoo is the commercially available Noble Formula Zinc Shampoo manufactured by Ontos, Inc (Chehalis, Wash.). The shampoo comprises zinc pyrithione 2% prepared with purified water, sodium laureth sulfate, sodium laurel sulfate, glycol stearate, propylene glycol, sodium chloride, methyl paraben, hydroxyethelcellulose, and propyl paraben. The shampoo is applied to the subject daily as prophylactic treatment for the prevention of Parkinson's disease. The subject is followed up with clinical exam visits semi-annually over 7 years with no symptoms of Parkinson disease present on examination for the duration of follow-up.

### Example 3

A 49-year old male patient is clinically evaluated by a physician for deterioration of health because of his progression of symptoms of Parkinson's disease which include tremor, speech dysfunction, aberrant personality changes, abulia, and difficulties with mentation. Patient has no overt symptoms of Malassezia infection. However, clinical examination reveals that patient has anosmia and nasal sinusitis. Clinical laboratory testing is performed with results positive for Malassezia infection demonstrated by microscopy morphologic findings and positive growth on Dixon agar media plating (glycerol mono-oleate, or Sabouraud dextrose agar, covered with a layer of olive oil and incubated at 37° C.), with identification further confirmed by matrix-assisted laser desorption ionization—time of flight mass spectrometry and multiplex polymerase chain reaction. Patient is treated with an intravenous administration of a commercially available amphotericin formulation of amphotericin B deoxycholate at 1.0 mg/kg/day in combination with a twice daily treatment regimen of intranasal aqueous saline solution. The solution comprises a mixture of 1 teaspoon of baking soda, 1.5 teaspoons of salt, and 1 quart sterilized distilled water. The solution is administered intranasally using a pulsating irrigation device. The pulsating irrigation device sends a gentle pulse of the solution into the nose via a nasal irrigation tip that can reach into and contact with the upper nasal passages. The amphotericin B treatment regimen is discontinued after 3 weeks. The total amount of amphotericin B administered to the patient over the course of treatment is about 1.8 grams. The nasal wash is continued to be administered to the patient indefinitely on a once daily basis. Routine follow-up is conducted by the physician 6 months after completion of the amphotericin B treatment course. Progression of Parkinson's disease is halted in the patient as measurement with the MDS-UPDRS. Patient's caregiver reports that patient has “returned to being his normal self again” with improved mood, improved affect, decreased fatigue, decreased abulia, and improved mentation. Patient self-reporting of amelioration of symptoms is consistent with caregiver reports.

### Example 4

### Case Study

The subject of the case study was a genotypic male, approximately age 50, who received a formal clinical diagnosis of idiopathic Parkinson disease approximately 5 years ago. The subject displayed classical signs and symptoms of Parkinson disease including masked facies, cogwheel rigidity, pill-rolling tremor, and bradykinesia. The subject described a complete anosmia (i.e., the inability to perceive odor or a lack of functioning olfaction). The subject had no known uncontrolled medical conditions (e.g., known active hepatic disease, inadequately treated hypertension, congestive heart failure). The subject was not taking carbidopa-levodopa therapy or any other dopaminergic replacement pharmacologics. The subject had no known contraindication or allergy to fluconazole or zinc pyrithione in any form. On Day 1, the subject orally ingested fluconazole 400 mg (2 capsules); subsequently, the subject orally ingested 200 mg (1 capsule) a day for the remainder of a 3-month treatment period. The subject also washed his head, neck, and shoulders every third day with a shampoo containing 1% zinc pyrithione for the entire 3-month treatment period.

Results: The subject reported complete restoration of his sense of smell during week 4 of treatment. The restoration of his sense of smell continued for duration of treatment. The subject reported no worsening of symptoms as measured by the Movement Disorder Society—Unified Parkinson's Disease Rating Scale (MDS-UPDRS) over the 3-month treatment duration. Further treatment for an additional 1 month beyond the 3-month treatment period resulted in improved mood during week 4 of treatment. The improved mood continued for the duration of treatment.

### Example 5

### Randomized Double-Blind Placebo-Controlled Pilot Study of Fluconazole Plus Zinc Pyrithione in Patients with Parkinson's Disease

This study tests whether 3 months of treatment with combination of fluconazole and zinc pyrithione alters disease symptoms in patients with Parkinson's Disease. The patient population is comprised of patients of either sex, ages 30 to 80, with idiopathic Parkinson's Disease with a modified Hoehn and Yahr stage 3 or less. Patients may be on stable doses of carbidopa-levodopa therapy for at least 1 month prior to baseline and expected to maintain a stable dose for the duration of the study. Patients with uncontrolled medical conditions (e.g., known active hepatic disease, inadequately treated hypertension, congestive heart failure) are excluded. Patients with known contraindication or allergy to fluconazole or zinc pyrithione in any form are excluded. Each treatment group is comprised of 12 subjects.

Optionally, the environment is sanitized by removing any humidifiers. In addition, further environmental sanitation is performed by replacing (preferred) or washing pillow(s), pillow-case(s), bed sheets, blankets, towels, undergarments, socks, hats, etc. at least twice weekly. An air purifier with HEPA filter is present in the bedroom (e.g., GERMGUARDIAN® AC4825 Purifier).

Patients are assigned treatment from a predetermined randomization schedule. Half the patients are instructed to shampoo every third day with a shampoo containing 1% zinc pyrithione from an unlabeled bottle and avoid all other shampoos. These same patients also take active fluconazole that has been over-encapsulated. The other half of patients are instructed to shampoo every third day with a shampoo not containing 1% zinc pyrithione in an unlabeled bottle and avoid all other shampoos. These same patients also take a capsule containing an inert substance.

Fluconazole capsules contain 200 mg of drug each. On Day 1, subjects take 400 mg (2 capsules); subsequently, they take 200 mg (1 capsule) a day for the remainder of the 3-month treatment period. The shampoo contains zinc pyrithione 1%. Subjects are instructed to shampoo every third day (or at least twice weekly) for the entire 3-month treatment period.

Outcome measures are determined using The Movement Disorder Society—Unified Parkinson's Disease Rating Scale (MDS-UPDRS) which is administered at baseline and then monthly for the 3-month treatment period. All parts of the MDS-UPDRS assessed.

All data is summarized and depicted graphically. Simple descriptive statistics (e.g., mean, standard deviation) may be calculated.

Results. Patient examination at the end of the 3-month treatment period demonstrates an MDS-UPDRS score that is improved or maintained as same. Of significant note is that no further worsening of patient's symptoms occurs, even over much longer periods of time. Improvement or further improvement in MDS-UPDRS score may occur over a much longer period of time, such as between 1 and 2 years. After the 3 months of protocol treatment, however, the pathogenic version of the Malassezia is fully eradicated.

### Example 6

### Case Study—Topical Anti-Malassezial Treatment

Subject is a 70-year-old male suffering from Parkinson's disease. Patient reported anosmia prior to treatment.

Beginning on Day 1, the subject washed his head with shampoo containing 2% ketoconazole. On Day 14, subject switched shampoo to a shampoo containing 2% zinc pyrithione. Subject paid particular attention to the danger triangle and also washed his scalp, temples, cheeks, forehead, neck and shoulders, and upper back. Patient did not wash inside his nasal passages. Patient did apply shampoo immediate under his nose and philtrum area. Patient applied the shampoo 4 to 5 times a week for the duration of the treatment period. During the treatment period, the subject did not take an oral anti-malassezial agent.

Results: The subject reported partial restoration of his sense of smell during week 5 of the two-month treatment period. Subject reports that the first odors he had noticed during week 5 were cooking odors. Subject reports restored ability to smell food on a plate which correlates with the benefits of improved appetite and increased gustatory hedonia. The partial restoration of his sense of smell continued for duration of treatment. The subject reported improved affect and no worsening of symptoms as measured by self-reporting over the 2-month treatment duration.

### Example 7

### Case Study—Combination of Topical and Oral Anti-Malassezial Treatment

Subject is a 72-year-old female suffering from Parkinson's disease. Patient reported anosmia prior to beginning combined oral and topical anti-malassezial treatment. Patient began treatment in early January 2018. Relevant excerpts from interviewing subject (dated Feb. 6, 2018):

On Day 1, subject took 400 mg of fluconazole by mouth; subsequently, subject took 200 mg of fluconazole a day. On Day 1, and subsequent days, subject used shampoo containing zinc pyrithione 1%. Specifically, subject washed her head, neck, and shoulders area every second or third day with a shampoo containing zinc pyrithione 1%.

Results: approximately four to six weeks into the 3-month treatment period, the subject reported that she had determined she regained her sense of smell. Subject specifically made note of her new appreciation for food and dining odors that was previously absence from olfactory sensation by subject. The subject reported improved affect and no worsening of symptoms as measured by self-reporting.

### Example 8

Case Study—Combination of Topical and Oral Anti-Malassezial Treatment

Subject is a 51-year-old male suffering from Parkinson's disease. Patient reported complete anosmia (i.e., no sense of smell) prior to beginning combined oral and topical anti-malassezial treatment. Patient began treatment in early July 2017. Relevant excerpts from interviewing subject (dated Aug. 7, 2017 and Aug. 16, 2017):

On Day 1, subject took 400 mg of fluconazole by mouth; subsequently, subject took 200 mg of fluconazole a day for the duration of the treatment. On Day 1, and subsequent days of the treatment, subject used shampoo containing zinc pyrithione 1%. Specifically, subject washed his head, neck, and shoulders area every day with a shampoo containing zinc pyrithione 2%.

Results: Approximately 3 and a half weeks after he began treatment, the subject reported that experienced a complete restoration of his sense of smell. Subject specifically made note of his restored ability to smell fried onions in a kitchen and also his restored ability to smell to detect dog feces while outside on a hiking trail.

The restoration of his sense of smell continued for duration of treatment. The subject reported no worsening of symptoms by self-reporting and as measured by the Movement Disorder Society—Unified Parkinson's Disease Rating Scale (MDS-UPDRS) over the 3-month treatment duration. Further treatment for an additional 1 month beyond the 3-month treatment period resulted in improved mood (with correlative positive affect) that began during week 4 of treatment. The improved mood continued for the duration of treatment period.

These examples are intended to provide non-limiting examples that may occur in relation to the present invention.

Those skilled in the art will recognize or be able to ascertain using no more than routine experimentation, numerous equivalents to the specific embodiments of the invention described herein. Such equivalents are intended to be encompassed by the present disclosure.

The entire contents of all references, patents, and patent applications cited herein are expressly incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for preventing, treating or mitigating Parkinson's disease in a subject that is in need of such prevention, treatment or mitigation, the method comprising administering to the subject an anti-malassezial agent.

2. The method according to claim 1, wherein the method comprises administering to the subject an anti-malassezial agent containing an anti-fungal compound.

3. The method according to claim 2, wherein the anti-fungal compound is selected from the group consisting of zinc pyrithione, ciclopirox olamine, a polyene, an azole, an allylamine, a morpholine, a sulfonamide, a glucan synthesis inhibitor, and mixtures thereof.

4-8. (canceled)

9. The method according to claim 1, wherein the method comprises administering to the subject an anti-malassezial agent containing an anti-inflammatory compound.

10. The method according to claim 9, wherein the anti-inflammatory compound is selected from the group consisting of a steroidal anti-inflammatory drug, a non-steroidal anti-inflammatory drug, and a topical calcineurin inhibitor.

11-13. (canceled)

14. The method according to claim 1, wherein the method comprises administering to the subject an anti-malassezial agent containing an antiseptic compound.

15. The method according to claim 14, wherein the antiseptic compound is formulated as a laundry composition.

16. A method for preventing, treating or mitigating Parkinson's disease in a subject that is in need of such prevention, treatment or mitigation, the method comprising administering to the subject UV light phototherapy.

17. (canceled)

18. A pharmaceutical composition comprising an anti-malassezial agent and a pharmaceutically-acceptable carrier for use in preventing, treating or mitigating Parkinson's disease in a subject that is in need of such prevention, treatment or mitigation.

19. (canceled)

20. A method for reducing the destruction of nigral dopaminergic neurons in a subject, the method comprising administering to the subject a compound containing an anti-malassezial agent.

21-29. (canceled)

30. The method of claim 2, wherein the anti-fungal agent is a lipase inhibitor.

31-35. (canceled)

36. The method of claim 1, wherein the anti-malassezial agent is oteseconazole, tavaborole or 2-(3,5-dimethyl-1H-pyrazol-1-yl)-5-methylphenol.

37. (canceled)

38. The method of claim 1, wherein the anti-malassezial agent is co-administered in combination with a different medication for treating Parkinson's disease selected from the group consisting of: carbidopa-levodopa, pramipexole, ropinirole and rotigotine, apomorphine, selegiline, rasagiline, entacapone, tolcapone, bromocriptine, benztropine, trihexyphenidyl, and amantadine.

39. (canceled)

40. A method for treating olfactory dysfunction in a patient in need of treatment comprising topical administration of an anti-malassezial agent to the danger triangle of the face and optionally comprising administration to the inside of the nasal passages and optionally comprising administration to the entire head and shoulders.

41-46. (canceled)

47. A method for treating Parkinson's Disease or a symptom of Parkinson's disease in a patient in need of treatment comprising topical administration of an anti-malassezial agent to the danger triangle of the face and optionally comprising administration to the inside of the nasal passages and optionally comprising administration to the entire head and shoulders.

48-53. (canceled)
